(19) United States
(12) Patent Application Publication
(10) Pub. No.: US 2023/0270663 A1
(43) Pub. Date: Aug. 31, 2023

(54) FORMULATION FOR THE TREATMENT OF FINE LINES AND WRINKLES AND USES THEREOF

(71) Applicant: RoC OPCO LLC, New York, NY (US)

(72) Inventors: Arthur Pellegrino, New York, NY (US); Ellen Delisle, Hauppauge, NY (US); Lea Benkoski, Newtown, PA (US)

(21) Appl. No.: 18/176,242

(22) Filed: Feb. 28, 2023

Related U.S. Application Data
(60) Provisional application No. 63/314,806, filed on Feb. 28, 2022.

(51) Int. Cl. A61K 8/9789; A61K 8/73; A61K 8/67; A61K 8/49; A61Q 19/08

(52) U.S. Cl. CPC A61K 8/9789; A61K 8/735; A61K 8/671; A61K 8/676; A61K 8/496; A61Q 19/08

(57) ABSTRACT

Disclosed herein are cosmetic compositions comprising Swertia chirata extract, hyaluronic acid, and retinoid, and a method of reducing fine lines and/or wrinkles, and/or smoothing the skin by topically applying the cosmetic compositions to the skin.

### BACKGROUND

This application claims the benefit of priority from U.S. Patent Application No. 63/314,806, filed on Feb. 28, 2022, the disclosure of which is incorporated herein by reference in its entirety.

Disclosed herein are cosmetic compositions containing Swertia chirata extract, hyaluronic acid, and retinoid, which work together to improve the appearance of fine lines and wrinkles, and smooth the skin.

### SUMMARY

One general aspect of the invention includes a cosmetic composition which includes Swertia chirata extract, hyaluronic acid, and retinoid. In this embodiment the composition can include about 0.005 wt. % to about 0.500 wt. % Swertia chirata extract. In this embodiment, the composition can include about 0.005 wt. % to about 1.00 wt. % retinoid. In this embodiment the composition can include about 0.001 wt. % to about 2.0 wt. % hyaluronic acid.

In an embodiment of the invention the cosmetic composition includes about 0.005 wt. % to about 0.500 wt. % Swertia chirata extract; about 0.005 wt. % to about 1.00 wt. % retinoid; and about 0.001 wt. % to about 2.0 wt. % hyaluronic acid. In a preferred embodiment, the composition includes about 0.030 wt. % Swertia chirata extract; about 0.070 wt. % retinoid; and about 0.050 wt. % hyaluronic acid. In another preferred embodiment, the composition includes about 0.030 wt. % Swertia chirata extract; about 0.110 wt. % retinoid; and about 0.002 wt. % hyaluronic acid.

In an embodiment of the invention, the retinoid is selected from retinol, retinal, tretinoin, isotretinoin, alitretinoin, etretinate acitretin, adapalene, bexarotene, and tazarotene, and trifarotene. In a preferred embodiment, the retinoid is retinol.

In an embodiment of the invention, the hyaluronic acid is a mixture of hyaluronic acids of different molecular weights. In this embodiment, the mixture can contain at least three different hyaluronic acids of different molecular weights.

In an embodiment of the invention, the cosmetic composition can also include a vitamin C compound. In a preferred embodiment, the vitamin C compound can be ascorbic acid or 3-O-ethyl ascorbic acid.

In an embodiment of the invention, the cosmetic composition can also include a chromenone of formula (I):

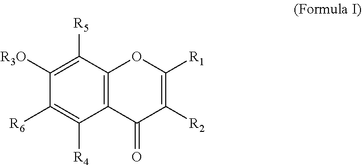

or a salt thereof; wherein:
R¹ and R² are identical or different, and are selected from the group consisting of H, —(C═O)—R⁷, —C(═O)—OR⁷, a straight-chain or branched C₁- to C₂₀-alkyl group, wherein the alkyl is optionally at least once interrupted by oxygen, a straight-chain or branched C₃- to C₂₀-alkenyl group, a straight-chain or branched C₁- to C₂₀-hydroxyalkyl or di- or polyhydroxyalkyl group, where the hydroxyl group is bonded to a primary or secondary carbon atom of the alkyl, and wherein the alkyl is optionally at least once interrupted by oxygen, a C₃- to C₁₀-cycloalkyl group and a C₃- to C₁₂-cycloalkenyl group (where the cyclic group is optionally bridged by —CH₂)ₙ— group where n=1 to 3);
R³ is H or a straight-chain or branched C₁- to C₂₀-alkyl group;
R⁴ is H or —OR⁸;
R⁵ and R⁶ are identical or different, and are selected from the group consisting of H or
—OH, a straight-chain or branched C₁- to C₂₀-alkyl group (wherein the alkyl is optionally at least once interrupted by oxygen), a straight-chain or branched C₃- to C₂₀-alkenyl group, and a straight-chain or branched C₁- to C₂₀-hydroxyalkyl group, where the hydroxyl group is bonded to a primary or secondary carbon atom of the alkyl, and wherein the alkyl is optionally at least once interrupted by oxygen;
R⁷ is selected from the group consisting of H, a straight-chain or branched C₁- to C₂₀-alkyl group, wherein the alkyl is optionally at least once interrupted by oxygen, a straight-chain or branched C₃- to C₂₀-alkenyl group, and a straight-chain or branched C₁- to C₂O-hydroxy-alkyl or a di- or polyhydroxyalkyl group, where the hydroxyl group is bonded to a primary or secondary carbon atom of the alkyl and wherein the alkyl is optionally at least once interrupted by oxygen; and
R⁸ is H or a straight-chain or branched C₁- to C₂₀-alkyl group.

In this embodiment, the cosmetic composition includes about 0.1 wt. % to about 2.0 wt. % of a compound of formula (I). In a preferred embodiment, the compound of formula (I) is dihydroxy methylchromenone.

In an embodiment of the invention, the cosmetic composition includes at least 60 wt. % water.

In an embodiment of the invention, the cosmetic composition can be a cream or a serum.

Another general aspect of the invention includes a method of reducing fine lines and/or wrinkles, and/or smoothing the skin, by topically administering to the skin the cosmetic composition. In this embodiment, the skin can be skin of the neck and/or face. In this embodiment, the cosmetic composition can be applied to the forehead, the lip, between the brows, under the eyes, the nasolabial folds, crow's feet, or a combination thereof. In this embodiment, the skin can be cleansed skin. In this embodiment, the cosmetic composition can be applied in the morning, the evening, or both.

### BRIEF DESCRIPTION OF THE FIGURES

FIG. 1 depicts the improvement in fine lines and wrinkles between the brows; (A) shows the skin before application of the cosmetic composition; and (B) shows the skin immediately after application of the cosmetic composition.

FIG. 2 depicts the improvement in fine lines and wrinkles of the forehead; (A) shows the skin before application of the cosmetic composition; and (B) shows the skin after 4 weeks of daily application of the cosmetic composition.

FIG. 3 depicts questionnaire results for subjects administered a cosmetic composition according to a method disclosed herein.

FIG. 4 depicts questionnaire results for subjects administered a cosmetic composition according to a method disclosed herein.

FIG. 5 depicts questionnaire results for subjects administered a cosmetic composition according to a method disclosed herein.

FIG. 6 depicts questionnaire results for subjects administered a cosmetic composition according to a method disclosed herein.

### DETAILED DESCRIPTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

Unless otherwise indicated, all parts and percentages are by weight. As used herein, the term “about” refers to plus or minus 10% of the indicated value. Unless otherwise stated or made clear by context, weight percentages are provided based on the total amount of the composition in which they are described. As used herein, the singular forms “a,” “an,” and “the” include plural referents unless the context clearly dictates otherwise.

Described herein are cosmetic compositions containing Swertia chirata extract, hyaluronic acid, and a retinoid. Also disclosed herein are methods of improving the appearance of fine lines and wrinkles and smoothing the skin by topically applying the cosmetic compositions to the skin.

Swertia Chirata Extract

Swertia chirata, also known as Indian gentian, is a traditional Ayurvedic herb with 4-angled flowers of greenish yellow color tinged with purple. It is native to temperature Himalayas and found on high altitude hills. The main component of extracts contains swertiamarin, which belongs to the iridoid family of molecules typically found in medicinal plants.

Swertia chirata extract stimulates keratinocyte proliferation to regenerate thin epidermis and improve the look of aged and wrinkled skin. It has been found to have anti-aging properties through a biomimetic pathway on the reduction of the appearance of wrinkles, especially vertical wrinkles. The mechanism of action is based on stem cell therapy with a cell-to-cell communication between adipose-derived stem cells (ADSC) and keratinocytes through growth factor action to auto-regenerate skin; Swertia chirata releases these growth factors which are proven to promote keratinocyte proliferation, resulting in a thicker epidermis with a higher number of keratinocytes layers. The resulting thicker epidermis fills in existing wrinkles for a resurfacing effect.

In exemplary embodiments, the Swertia chirata extract is a Swertia chirata leaf extract. Exemplary embodiments of the Swertia chirata extract contain relatively high amounts of swertiamarin, for example at least 80%, at least 85% or at least 90% swertiamarin. As an alternative to a Swertia chirata extract, swertiamarin can be added. Accordingly, as used in compositions described herein, Swertia chirata extract and swertiamarin are used interchangeably and the component can be added as a Swertia chirata extract or as swertaimarin.

In embodiments of the invention, the cosmetic compositions contain about 0.005 wt. % to about 0.5 wt. % Swertia chirata extract (or swertiamarin). For example, the compositions can contain about 0.005 wt. % to about 0.10 wt. %, 0.01 wt. % to about 0.1 wt. %, about 0.005 wt. % to about 0.050 wt. %, or 0.015 wt. % to about 0.040 wt. % Swertia chirata extract (or swertiamarin). In exemplary embodiments, the compositions can contain about 0.005 wt. %, about 0.010 wt. %, about 0.015 wt. %, about 0.020 wt. %, about 0.025 wt. %, about 0.030 wt. %, about 0.035 wt. %, about 0.040 wt. %, about 0.045 wt. %, or about 0.050 wt. % Swertia chirata extract (or swertiamarin). In a preferred embodiment, the compositions contain about 0.030 wt. % Swertia chirata extract (or swertiamarin).

An exemplary source of Swertia chirata extract is SWT-7™ available from Lucas Meyer Cosmetics (Paris, FR). SWT-7 is a formulation containing Swertia chirata extract containing high amounts of swertiamarin and titrated to 1.25% swertiamarin. SWT-7™ is sold as a hydrosoluble powder (SWT-7™ H) and as a liposoluble liquid (SWT-7™ L). In exemplary embodiments of the invention, Swertia chirata extract is added as SWT™-7 H. This Swertia chirata extract is also described in U.S. Patent Application Publication No. 2017/0020796, which is incorporated herein by reference in its entirety.

Hyaluronic Acid

According to the invention, the terms “hyaluronic acid” and “HA” refer to both the free acid as well as any pharmaceutically acceptable salts thereof, for example sodium hyaluronate. Pharmaceutically acceptable salts include, but are not limited to, alkali metal salts such as potassium hyaluronate and sodium hyaluronate.

According to the invention, the HA can come from any source, including, but not limited to, synthetic sources and natural sources. Traditionally, HA was extracted from rooster combs. However, this traditional method faces growing concern over the use of animal-derived components in biomedical, pharmaceutical, and cosmetic applications. HA can also be produced via streptococcal fermentation which has lower production costs and produces less environmental pollution, and produces a mixture of HA of different molecular weights. Other recent HA production processes include recombinant production from gram-positive and gram-negative bacteria, such as Bacillus sp., Lactococcos lactis, Agrobacterium sp., and Escherichia coli. Further, recombinant microorganisms like Bacilli and Escherichia coli are endotoxin-free, making them a safer, alternative HA production source. HAs with different molecular weights can be obtained by selective production methods that generate particular molecular weight ranges or by controlled hydrolysis of higher molecular weight HAs.

According to the invention, the HA used in the composition can be linear, cross-linked or crosslinked with other components. Typically, HA has a molecular weight of from about 1 kDa to about 20,000 kDa. In an embodiment of the invention, the compositions contain one or more HA with a molecular weight of about 1 kDa to about 2000 kDa. The HA can be present in a singular molecular weight range or as a mixture of HAs with different molecular weight ranges. HA can have a low molecular weight range, for example less than about 50 kDa, a mid-molecular weight range, for example from about 50 kDa to about 1000 kDa, or a high molecular weight range, for example greater than about 1000 kDa. The HA used in compositions or the invention can be within a singular molecular weight range, i.e., a low molecular weight range HA, a mid-molecular weight range HA, or a high molecular weight range HA, or can be a mixture of two or more of low molecular weight range HA, mid-molecular weight range HA, and high molecular weight range HA. HA of differing molecular weight ranges can be added as individual commercially available compositions of a specified molecular weight range which may be added individually or as a premix, or can be provided as a commercially available mixture of HA having different molecular weights. Preferred embodiments of the invention use a mixture of low molecular weight range HA, mid-molecular weight range HA, and high molecular weight range HA.

An exemplary source of HA is BP-Triluronic™ Acid, which is a combination of three different HA's of different molecular weights: 1) a high molecular weight HA having a molecular weight of about 1500 kDa to about 2000 kDa; 2) a mid-molecular weight HA having a molecular weight of about 300 kDa to about 500 kDa; and 3) a low molecular weight HA having a molecular weight of about 12 kDa to about 15 kDa.

Another exemplary source of HA is HyaCare® Filler CL, which is a cross-linked HA with a 3D network structure.

Another exemplary source of HA is EES Cosmetic Solution's hyaluronic acid which contains HAs with molecular weights ranging from about 5 kDa to about 11 kDa.

In embodiments of the invention, the cosmetic compositions contain about 0.001 wt. % to about 2.0 wt. % HA from one or more sources. In embodiments of the invention, the composition contains about 0.001 wt. % to about 2.0 wt. %, about 0.001 wt. % to about 1.5 wt. %, about 0.001 wt. % to about 1.0 wt. %, about 0.001 wt. % to about 0.5 wt. %, about 0.001 wt. % to about 0.1 wt. %, about 0.001 wt. % to about 0.075 wt. %, or about 0.001 wt. % to about 0.050 wt. % HA. In other embodiments of the invention, the composition contains about 0.001 wt. % to about 0.050 wt. % HA. In embodiments of the invention, the compositions contain about 0.001 wt. % HA, about 0.005 wt. % HA, about 0.010 wt. % HA, about 0.015 wt. % HA, about 0.020 wt. % HA, about 0.025 wt. % HA, about 0.030 wt. % HA, about 0.035 wt. % HA, about 0.040 wt. % HA, about 0.045 wt. % HA, or about 0.050 wt. % HA. Preferred embodiments of the compositions contain about 0.002 wt. % HA or about 0.050 wt. % HA.

Retinoids

Retinoids are a class of chemical compounds that are derivatives and analogs of vitamin A, and are used in cosmetics and medicines to regulate epithelial cell growth to treat photoaging and skin wrinkles. Four generations of retinoids are generally recognized.
First generation retinoids such as retinol, retinal, tretinoin (i.e. retinoic acid or retin-A), isotretinoin, and alitretinoin.
Second generation retinoids such as etretinate and its metabolite acitretin.
Third generation retinoids such as adapalene, bexarotene, and tazarotene.
Fourth generation retinoids such as trifarotene.

Of the known retinoids, retinol is highly recommended by dermatologists for slowing the signs of skin aging and helping to maintain a youthful appearance. Retinol is a form of Vitamin A that naturally occurs in human skin. When employed regularly in a topical treatment, it can help improve skin firmness, visibly reduce the appearance of fine lines and deep wrinkles—and even help minimize the look of crow's feet and dark circles in the eye area.

Any retinoid can be used in the disclosed compositions and methods, with retinol being preferred.

In embodiments of the invention, the cosmetic compositions contain about 0.005 wt. % to about 1.00 wt. % of retinoid; about 0.010 wt. % to about 0.50 wt. % of retinoid; about 0.050 wt. % to about 0.150 wt. % of retinoid; or about 0.070 wt. % to about 0.110 wt. % of retinoid. In embodiments of the invention, the compositions contain about 0.050 wt. % retinoid, about 0.060 wt. % retinoid, about 0.070 wt. % retinoid, about 0.080 wt. % retinoid, about 0.090 wt. % retinoid, about 0.100 wt. % retinoid, about 0.110 wt. % retinoid, about 0.120 wt. % retinoid, about 0.130 wt. % retinoid, about 0.140 wt. % retinoid, or about 0.150 wt. % retinoid. In exemplary embodiments, the composition contains about 0.070 wt. % or about 0.110 wt. % retinoid. Preferably, the retinoid is retinol.

Cosmetic Compositions

In an embodiment, the cosmetic composition includes Swertia chirata extract (or swertiamarin), hyaluronic acid, and retinoid. The cosmetic compositions contain other active components, excipients, and solvents as described further below.

Certain chromenone derivatives have been shown to exhibit certain anti-aging effects. U.S. Pat. No. 8,518,986, the entire contents of which are incorporated by reference, teaches chromenone derivatives of formula (I):

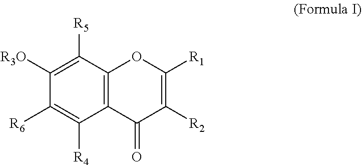

or a salt thereof where:
R¹ and R² are identical or different, and are selected from the group consisting of H, —(C═O)—R⁷, —C(═O)—OR⁷, a straight-chain or branched C₁- to C₂₀-alkyl group, wherein the alkyl is optionally at least once interrupted by oxygen, a straight-chain or branched C₃- to C₂₀-alkenyl group, a straight-chain or branched C₁- to C₂O-hydroxyalkyl or a di- or polyhydroxyalkyl group, where the hydroxyl group is bonded to a primary or secondary carbon atom of the alkyl, and wherein the alkyl is optionally at least once interrupted by oxygen, a C₃- to C₁₀-cycloalkyl group and a C₃- to C₁₂-cycloalkenyl group (where the cyclic group is optionally bridged by —CH₂)ₙ— group where n=1 to 3);
R³ is H or a straight-chain or branched C₁- to C₂₀-alkyl group;
R⁴ is H or —OR⁸;
R⁵ and R⁶ are identical or different, and are selected from the group consisting of H or —OH, a straight-chain or branched C₁- to C₂₀-alkyl group (wherein the alkyl is optionally at least once interrupted by oxygen), a straight-chain or branched C₃- to C₂₀-alkenyl group, and a straight-chain or branched C₁- to C₂₀-hydroxyalkyl group, where the hydroxyl group is bonded to a primary or secondary carbon atom of the alkyl, and wherein the alkyl is optionally at least once interrupted by oxygen;
R⁷ is selected from the group consisting of H, a straight-chain or branched C₁- to C₂₀-alkyl group, wherein the alkyl is optionally at least once interrupted by oxygen, a straight-chain or branched C₃- to C₂₀-alkenyl group, and a straight-chain or branched C₁- to C₂O-hydroxy-alkyl or a di- or polyhydroxyalkyl group, where the hydroxyl group is bonded to a primary or secondary carbon atom of the alkyl and wherein the alkyl is optionally at least once interrupted by oxygen; and
R⁸ is H or a straight-chain or branched C₁- to C₂₀-alkyl group.

Chromenone of formula (I) effectively potentiate the topical efficacy of retinoids. Preferred compounds of formula (I) for use in the methods, HA compositions, or retinoid compositions of the invention include compounds 1-11 shown below:

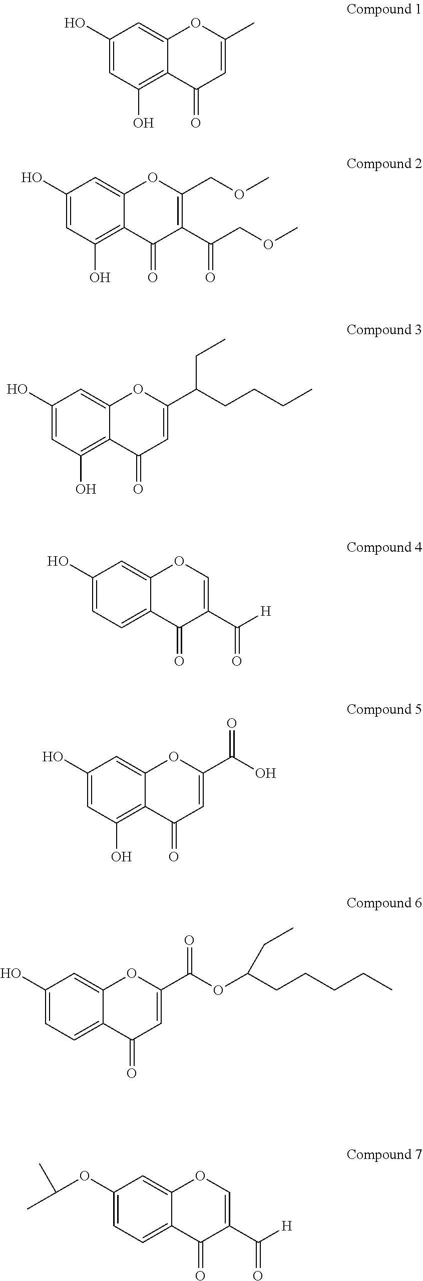

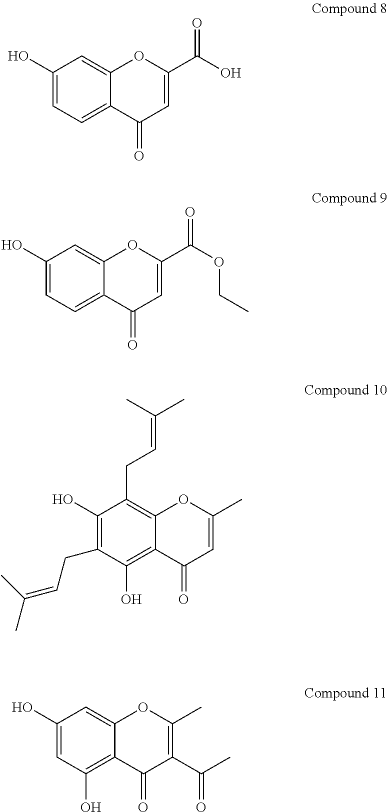

In an embodiment, the cosmetic composition can further comprise a chromenone of formula (I). When a chromenone of formula (I) is used, the amount of water is reduced by an amount corresponding to the amount of chromenone added. When present, the cosmetic compositions contain about 0.1 wt. % to about 2.0 wt. % of a chromenone of formula (I). For example, the cosmetic compositions can contain about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. % about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2.0 wt. % of a chromenone of formula (I). Preferably, when present, the cosmetic compositions contain about 0.1 wt. % of a chromenone of formula (I). Preferably, the chromenone of formula (I) is one of compound 1-11 above. Most preferably, the chromenone is the compound 1, also known as dihydroxy methylchromenone

In any embodiment, the composition may include a Vitamin C compound such as Vitamin C, analogs, derivatives and precursors that form Vitamin C or function similarly to Vitamin C in a cosmetic formulation. Vitamin C compounds may be added directly or as an extract known to be rich in vitamin C compounds. Exemplary Vitamin C compounds include 3-O-ethyl ascorbic acid; ascorbic acid; ascorbyl isostearate; ascorbyl glucoside; ascorbyl palmitate; magnesium ascorbyl phosphate; sodium ascorbyl phosphate; trisodium ascorbyl palmitate phosphate; ascorbyl methylsilanol pectinate; aminopropyl ascorbyl phosphate; potassium ascorbyl tocopheryl phosphate; ascorbyl tetraisopalmitate; tetrahexyldecyl ascorbate; and 3-glyceryl ascorbate. Extracts known to be rich in vitamin C compounds include, but are not limited to extracts of Terminalia ferdinandiana (i.e., Kakadu plum); Myrciaria dubia (i.e., camu-camu); Malpighia emarginata (i.e., acerola cherry); Averrhoa carambola (i.e., carambola/starfruit); and rose hip. Other suitable compounds useful as Vitamin C compounds include free radical scavenging agents, oxygen scavenging agents, and chelating agents.

The Vitamin C compound can be present in an amount of from about 0.01% up to about 30% by weight, for example, from about 0.1% to about 5% by weight, or about 1% by weight

Any embodiment of the invention can include one or more solvents and dermatological excipients known to be useful in the manufacture of cosmetic compositions. Excipients can include
a. Emulsifiers, including nonionic, cationic, anionic, or polymeric emulsifiers.
b. Rheology modifiers.
c. Humectants.
d. Surfactants, including, non-ionic, cationic, and anionic surfactants.
e. Emollients.
f. pH modifiers and buffers.
g. Antimicrobial agents.
h. Aromas including fruit or plant extracts, for example in the form of fragrances or essential oils.
i. Additional antioxidants.
j. Additional skin care antiaging/anti-wrinkle agents.
k. Film-forming agents.
l. FD&C colors

Embodiments of the invention also include solvents. In exemplary embodiments, more than about 60% of the composition comprises one or more solvents. Solvents include water and water soluble solvents, and water immiscible solvents. Water and water soluble solvents include, for example alcohols such as ethanol propanol, isopropanol, glycerin, and mixtures thereof. Water immiscible solvents include oils and waxes. As used herein, an oil is a water insoluble solvent such as mineral oil, vegetable oils and silicone oils, such as dimethicone and cyclomethicone. In exemplary embodiments, the composition includes water and/or water soluble solvents, and oils and/or water immiscible solvents.

According to the invention, the composition can be an emulsion, such as an oil-in-water emulsion or water-in-oil emulsion. The oil in the emulsion may be a carbon or hydrocarbon based oil or a silicone based oil, i.e. a silicone emulsion. The compositions can also be a solution, for example an aqueous solution, or a suspension in water or oil.

An exemplary oil-in-water emulsion contains about 60 wt %-90 wt % purified water and water soluble components and about 10 wt % to about 40 wt % components forming a water immiscible or oil phase. “Purified water” is water that does not contain ingredients which would be harmful to, or would cause adverse reactions to, the skin of a subject, such as a human. Distilled water and/or deionized water can be used.

In exemplary embodiments, the composition is a serum or cream. Serum, as used herein, refers to a product that is rapidly absorbed and penetrates into deeper layers of the skin. Serums typically have a light low viscosity, non-greasy finish and high concentrations of active ingredients.

In some embodiments, the composition comprises at least about 60% water by weight.

In some embodiments, the composition is an oil-in-water emulsion.

Uses

In one aspect, the invention discloses methods of reducing fine lines and wrinkles, and smoothing the skin is provided, by topically administering to the skin a cosmetic composition containing: Swertia chirata extract, hyaluronic acid, and retinoid, as described herein.

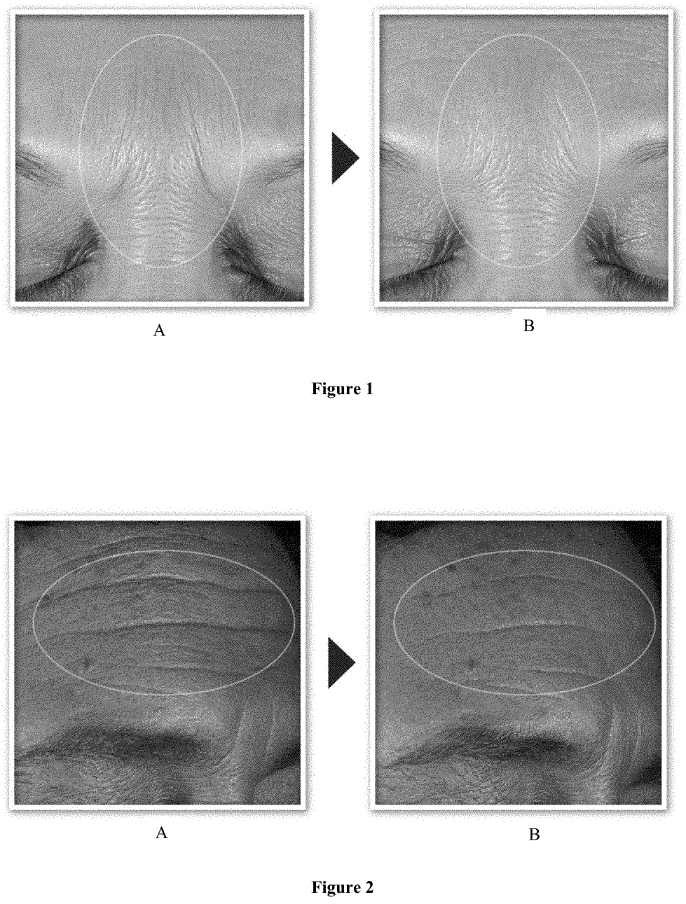

According to the present invention, the combination of Swertia chirata extract, hyaluronic acid, and retinoid work synergistically together to reduce the appearance of fine lines and wrinkles, including vertical wrinkles, also known as “11 lines”, as measured both immediately after application (i.e., within 5 minutes or after the product is absorbed into the skin), and over a longer period of time. Test subjects saw fine lines and wrinkles plumped and filled within approximately 5 minutes of application of the cosmetic compositions to the skin (see FIG. 1). After application over 4 weeks, test subjects had an improvement in the look of hard-to-treat wrinkles and had visible improvement in skin texture (see FIG. 2). In fact, after 8 weeks, test subjects saw 10 years of wrinkles effectively reversed. Additional advantages of the cosmetic compositions are their ability to quickly and effectively fill wrinkles while avoiding the use of needles and pain associated with typical injectable fillers. Further, injectable fillers are costly and require regular visits to licensed medical practitioners. Additionally, results of these fillers are entirely dependent on the skill of the practitioner, and therefore the results cannot consistently meet the patient's expectations. Accordingly, the cosmetic compositions can provide synergistic or complimentary benefits when used in combination with injectable fillers.

In some embodiments, fine lines and wrinkles that can be targeted for reduction include those of the forehead, lip, between the brows, under the eyes, nasolabial folds, and crow's feet.

In some embodiments, the cosmetic composition is applied to cleansed skin of the face and/or neck.

In some embodiments, the method entails applying the cosmetic composition in the morning (i.e. A.M.) and/or in the evening (i.e. P.M.).

In an exemplary embodiment the cosmetic composition of the invention is applied to the skin at least once, and preferably twice, per day. When applying two times per day, it is preferred to administer once in the morning, and once in the evening. The formulation is applied by massaging it on the skin with fingers. After application, the formulation is allowed to absorb into the skin.

While a single application has been found to improve skin properties, repeated use further improves results. Similarly, repeated application (daily or twice a day) extends the improvement in skin properties. Visible and measurable improvements in skin qualities are observed immediately after a single use, and increase when used twice daily for one week and twice daily for four weeks.

Additional Non-Limiting Exemplary Embodiments Include:
1. A cosmetic composition comprising Swertia chirata extract, hyaluronic acid, and retinoid.
2. The cosmetic composition of embodiment 1, wherein the composition comprises about 0.005 wt. % to about 0.500 wt. % Swertia chirata extract.
3. The cosmetic composition of embodiment 1 or 2, wherein the composition comprises about 0.005 wt. % to about 1.00 wt. % retinoid.
4. The cosmetic composition of any one of embodiments 1-3, wherein the composition comprises about 0.001 wt. % to about 2.0 wt. % hyaluronic acid.
5. The cosmetic composition of any one of embodiments 1-4, wherein the composition comprises about 0.005 wt. % to about 0.500 wt. % Swertia chirata extract; about 0.005 wt. % to about 1.00 wt. % retinoid; and about 0.001 wt. % to about 2.0 wt. % hyaluronic acid.
6. The cosmetic composition of any one of embodiments 1-5, wherein the composition comprises about 0.030 wt. % Swertia chirata extract; about 0.070 wt. % retinoid; and about 0.050 wt. % hyaluronic acid.
7. The cosmetic composition of any one of embodiments 1-6, wherein the composition comprises about 0.030 wt. % Swertia chirata extract; about 0.110 wt. % retinoid; and about 0.002 wt. % hyaluronic acid.
8. The cosmetic composition of any one of embodiments 1-7, wherein the retinoid is selected from the group consisting of retinol, retinal, tretinoin, isotretinoin, alitretinoin, etretinate acitretin, adapalene, bexarotene, and tazarotene, and trifarotene.
9. The cosmetic composition of any one of embodiments 1-8, wherein the retinoid is retinol.
10. The cosmetic composition of any one of embodiments 1-9, wherein the hyaluronic acid comprises a mixture of hyaluronic acids of different molecular weights.
11. The cosmetic composition of any one of embodiments 1-10, wherein the mixture comprises at least three hyaluronic acids of different molecular weights.
12. The cosmetic composition of any one of embodiments 1-11, further comprising a vitamin C compound.
13. The cosmetic composition of any one of embodiments 1-12, wherein the vitamin C compound comprises ascorbic acid or 3-O-ethyl ascorbic acid.
14. The cosmetic composition of any one of embodiment 1-13, further comprising a chromenone of formula (I):

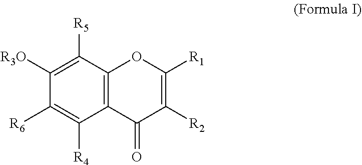

or a salt thereof; wherein:
R¹ and R² are identical or different, and are selected from the group consisting of H, —(C═O)—R⁷, —C(═O)—OR⁷, a straight-chain or branched C₁- to C₂₀-alkyl group, wherein the alkyl is optionally at least once interrupted by oxygen, a straight-chain or branched C₃- to C₂₀-alkenyl group, a straight-chain or branched C₁- to C₂₀-hydroxyalkyl or di- or polyhydroxyalkyl group, where the hydroxyl group is bonded to a primary or secondary carbon atom of the alkyl, and wherein the alkyl is optionally at least once interrupted by oxygen, a C₃- to C₁₀-cycloalkyl group and a C₃- to C₁₂-cycloalkenyl group (where the cyclic group is optionally bridged by —CH₂)ₙ— group where n=1 to 3);
R³ is H or a straight-chain or branched C₁- to C₂₀-alkyl group;
R⁴ is H or —OR⁸;
R⁵ and R⁶ are identical or different, and are selected from the group consisting of H or —OH, a straight-chain or branched C₁- to C₂₀-alkyl group (wherein the alkyl is optionally at least once interrupted by oxygen), a straight-chain or branched C₃- to C₂₀-alkenyl group, and a straight-chain or branched C₁- to C₂₀-hydroxyalkyl group, where the hydroxyl group is bonded to a primary or secondary carbon atom of the alkyl, and wherein the alkyl is optionally at least once interrupted by oxygen;
R⁷ is selected from the group consisting of H, a straight-chain or branched C₁- to C₂₀-alkyl group, wherein the alkyl is optionally at least once interrupted by oxygen, a straight-chain or branched C₃- to C₂₀-alkenyl group, and a straight-chain or branched C₁- to C₂₀-hydroxy-alkyl or -di- or polyhydroxyalkyl group, where the hydroxyl group is bonded to a primary or secondary carbon atom of the alkyl and wherein the alkyl is optionally at least once interrupted by oxygen; and
R⁸ is H or a straight-chain or branched C₁- to C₂₀-alkyl group.
15. The cosmetic composition of embodiment 14, wherein the cosmetic composition comprises about 0.1 wt. % to about 2.0 wt. % of a compound of formula (I).
16. The cosmetic composition of embodiment 14 or 15, wherein the compound of formula (I) is dihydroxy methylchromenone.
17. The cosmetic composition of any one of embodiments 1-16, wherein the cosmetic composition comprises at least 60 wt. % water.
18. The cosmetic composition of any one of embodiments 1-16, wherein the cosmetic composition is a cream or a serum.
19. A method of reducing fine lines and/or wrinkles, and/or smoothing the skin, comprising topically administering to the skin the cosmetic composition of any one of embodiments 1-18.
20. The method of embodiment 19, wherein the skin is the skin of the face and/or neck.
21. The method of embodiment 19, wherein the cosmetic composition is applied to the forehead, the lip, between the brows, under the eyes, the nasolabial folds, crow's feet, or a combination thereof
22. The method of any one of embodiments 19-21, wherein the skin is cleansed skin.
23. The method of any one of embodiments 19-22, wherein the cosmetic composition is applied in the morning, the evening, or both.
24. A method for improving skin properties in a subject, wherein the method comprises topically administering the cosmetic composition of any one of embodiments 1-18 to the subject in an amount sufficient to improve one or more skin properties.
25. The method according to embodiment 24, wherein the cosmetic composition is administered in an amount sufficient to decrease forehead fine lines and/or wrinkles by 5% to 50% as determined by image analysis.
26. The method according to embodiment 24 or 25, wherein the cosmetic composition is administered in an amount sufficient to decrease crow's feet fine lines and/or wrinkles by 5% to 50% as determined by image analysis.
27. The method according to any one of embodiments 24-26, wherein the cosmetic composition is administered in an amount sufficient to improve skin plumpness by 7% to 25% as determined by image analysis.
28. The method according to any one of embodiments 24-27, wherein the cosmetic composition is administered in an amount sufficient to increase skin firmness as determined by a 20% to 80% decrease in Cutometer R0 measurement.
29. The method according to any one of embodiments 24-28, wherein the cosmetic composition is administered in an amount sufficient to increase skin elasticity as determined by a 15% to 50% increase in Cutometer R2 measurement.
30. The method according to any one of embodiments 24-29, wherein the cosmetic composition is administered once.
31. The method according to any one of embodiments 24-29, wherein the cosmetic composition is administered twice per day for at least 1 week, at least 4 weeks, or at least 8 weeks.
32. A cosmetic composition according any one of embodiments 1-18 for use in improving one or more skin properties of a subject.
33. A cosmetic composition according any one of embodiments 1-18 for use in decreasing forehead fine lines and/or wrinkles by 5% to 50% as determined by image analysis.
34. A cosmetic composition according any one of embodiments 1-18 for use in decreasing crow's feet fine lines and/or wrinkles by 5% to 50% as determined by image analysis.
35. A cosmetic composition according any one of embodiments 1-18 for use in improving skin plumpness by 7% to 25% as determined by image analysis.
36. A cosmetic composition according any one of embodiments 1-18 for use in increasing skin firmness as determined by a 20% to 80% decrease in Cutometer R0 measurement.
37. A cosmetic composition according any one of embodiments 1-18 for use in increasing skin elasticity as determined by a 15% to 50% increase in Cutometer R2 measurement.

### EXAMPLES

The following examples are provided for illustrative purposes only and are intended to be purely exemplary of the disclosure and are not intended to limit the scope of the invention.

### Example 1

Table 1 is an exemplary general formula of the compositions of the invention. All weights are weight % unless otherwise specified

TABLE 1
GENERAL FORMULA

| Ingredient | Range | Preferred Range |
|---|---|---|
| Swertia Chirata Leaf Extract | 0.005-0.500 | 0.015-0.040 |
| Retinol | 0.005-1.00 | 0.050-0.150 |
| Hyaluronic Acid | 0.001-2.000 | 0.001-0.10 |
| Water, solvent, and excipients | q.s. | q.s. |
| Total | 100.00 | 100.00 |

### Example 2

Table 2 is an exemplary formula for a skin cream according to an embodiment of the invention. All weights are weight % unless otherwise specified.

TABLE 2
SKIN CREAM

| | | Weight % in |
|---|---|---|
| Ingredient | INCI Name | Formulation |
| PURIFIED WATER, USP | Water | 63.3000 |
| DUB PTO | Pentaerythrityl Tetraethylhexanoate | 6.5000 |
| USP GRADE KOSHER GLYCERIN | Glycerin | 6.0000 |
| Tegosoft E | PPG-15 Stearyl Ether | 3.9960 |
| | BHT | 0.0040 |
| IPSil 503 | Dimethicone Crosspolymer | 1.9317 |
| | Isododecane | 0.9659 |
| | Dimethicone | 0.4024 |
| Spectrastat PHL | Propanediol | 1.9500 |
| | 1,2-Hexanediol | 0.9000 |
| | Caprylhydroxamic Acid | 0.1500 |
| Element 14 PDMS 200 | Dimethicone | 2.2000 |
| SWT-7 H | Maltodextrin | 1.9703 |
| | Swertia Chirata Leaf Extract | 0.0297 |
| 1,3 Butylene glycol | Butylene Glycol | 2.0000 |
| PROMULGEN G | Stearyl Alcohol | 0.9855 |
| | Ceteareth-20 | 0.3645 |
| Compritol 888 CG ATO | Glyceryl Behenate | 1.1500 |
| PROMULGEN D | Cetearyl Alcohol | 0.8165 |
| | Ceteareth-20 | 0.3335 |
| Access White VCE PPT | 3-O-Ethyl Ascorbic Acid | 1.0000 |
| SEPIGEL 305 | Polyacrylamide | 0.3000 |
| | Water | 0.2588 |
| | C13-14 Isoparaffin | 0.1500 |
| | Laureth-7 | 0.0413 |
| Poly-Pore 120TRE | Allyl Methacrylates Crosspolymer | 0.2200 |
| | Polysorbate 20 | 0.1100 |
| | Retinol | 0.1100 |
| | Tocopherol | 0.1100 |
| SYMCALMIN | Pentylene Glycol | 0.2401 |
| | Butylene Glycol | 0.2401 |
| | Hydroxyphenyl Propamidobenzoic Acid | 0.0192 |
| | Ascorbyl Palmitate | 0.0005 |
| Aristoflex AVS | Sodium Acryloyldimethyltaurate/VP Crosspolymer | 0.4500 |
| COSMEDIA SP | Sodium Polyacrylate | 0.3520 |
| | Water | 0.0480 |
| BP-Triulonic Acid | Water | 0.1970 |
| | Sodium Hyaluronate | 0.0020 |
| | Phenoxyethanol | 0.0010 |
| 50% NaOH solution | Sodium Hydroxide | 0.0500 |
| | Water | 0.0500 |
| Disodium EDTA | Disodium EDTA | 0.1000 |

| | |
|---|---|
| Total | 100 |

### Example 3

Table 3 is an exemplary formula for a skin serum composition according to an embodiment of the invention. All weights are weight % unless otherwise specified.

TABLE 3
SKIN SERUM

| | | Weight % in |
|---|---|---|
| Ingredient | INCI Name | Formulation |
| PURIFIED WATER, USP | Water | 63.57100000 |
| DUB PTO | Pentaerythrityl Tetraethylhexanoate | 6.50000000 |
| Glycerin 99.7% USP | Glycerin | 4.98500000 |
| | Water | 0.01500000 |
| Canapeg 400 | PEG-8 | 4.00000000 |
| PPG-15 Stearyl Ether | PPG-15 Stearyl Ether | 4.00000000 |
| PROMULGEN G | Stearyl Alcohol | 1.64250000 |
| | Ceteareth-20 | 0.60750000 |
| PROMULGEN D | Cetearyl Alcohol | 1.59750000 |
| | Ceteareth-20 | 0.65250000 |
| SWT-7 H | Maltodextrin | 1.97029703 |
| | Swertia Chirata Leaf Extract | 0.02970297 |
| 1,3 Butylene glycol | Butylene Glycol | 2.00000000 |
| SEPIGEL 305 | Polyacrylamide | 0.80000000 |
| | Water | 0.69000000 |
| | C13-14 Isoparaffin | 0.40000000 |
| | Laureth-7 | 0.11000000 |
| XIAMETER PMX-0246 | Cyclohexasiloxane | 1.92000000 |
| CYCLOHEXASILOXANE | Cyclopentasiloxane | 0.08000000 |
| Mikrokill COS | Phenoxyethanol | 0.64000000 |
| | Caprylyl Glycol | 0.20000000 |
| | Chlorphenesin | 0.16000000 |
| REFINED SHEA BUTTER | Butyrospermum Parkii (Shea) Butter | 1.00000000 |
| Hyacare Filler CL | Water | 0.30500000 |
| | Ethylhexyl Stearate | 0.13500000 |
| | Sodium Hyaluronate Crosspolymer | 0.02000000 |
| | Polyglyceryl-4 | 0.02000000 |
| | Diisostearate/Polyhydroxystearate/Sebacate | |
| | Sodium Isostearate | 0.02000000 |
| Aristoflex AVS | Sodium Acryloyldimethyltaurate/VP Crosspolymer | 0.50000000 |
| SYMCALMIN | Pentylene Glycol | 0.24014423 |
| | Butylene Glycol | 0.24014423 |
| | Hydroxyphenyl Propamidobenzoic Acid | 0.01921154 |
| | Ascorbyl Palmitate | 0.00050000 |
| POLY-PORE 120RE | Allyl Methacrylates Crosspolymer | 0.20685000 |
| | Retinol | 0.07000000 |
| | Polysorbate 20 | 0.06300000 |
| | BHT | 0.01015000 |
| | Propyl Gallate | 0.00004900 |
| Tenox BHT | BHT | 0.10000000 |
| Disodium EDTA | Disodium EDTA | 0.10000000 |
| DIHYDROXYMETHYLCHROMONE | Dihydroxy Methylchromenone* | 0.10000000 |
| Sodium Hydroxide 20% solution | Water | 0.04000000 |
| | Sodium Hydroxide | 0.01000000 |
| BP-Triulonic Acid | Water | 0.04925000 |
| | Sodium Hyaluronate | 0.00050000 |
| | Phenoxyethanol | 0.00025000 |
| ASCORBIC ACID FINE POWDER | Ascorbic Acid | 0.05000000 |
| DL ALPHA TOCOPHERYL | Tocopheryl Acetate | 0.05000000 |
| ACETATE | | |
| ALLANTOIN | Allantoin | 0.05000000 |
| Hyaluronic Acid, 5-11 K Dalton | Hyaluronic Acid | 0.02900000 |

| | |
|---|---|
| Total | 100 |

### Example 4

Example 4 provides details regarding testing of the skin serum composition of Example 3.

Subject Selection: Subjects were enrolled in accordance with the following inclusion/exclusion criteria.

Inclusion Criteria
1. Females between the ages of 35 and 65 years (inclusive) in general good health (no physical required).
2. Individuals with a global facial fine lines and wrinkle on the forehead) score of “5” (noticeable) or greater (for qualification purposes only).
3. Individuals who indicated their willingness to participate in the study, follow directions, and remain on the study for the entire 8-week test period.
4. Individuals who could read understand and sign the Informed Consent Form.
5. Individuals who were regular users of retinol.

Exclusion Criteria
1. Women who were pregnant, planning a pregnancy, lactating and/or nursing a child.
2. Individuals with any visible skin disease.
3. Individuals with sunburn, suntan on the forearms or planning a vacation with sun-exposure or planning the use of a tanning booth during the course of the study.
4. Individuals engaged in a concurrent research project of a product involving the face/neck.
5. Individuals taking medications that might have interfered with the test results, including the use of steroidal/non-steroidal anti-inflammatory drugs or antihistamines, Accutane or any type of prescription acne medication.
6. Individuals with acne, active atopic dermatitis/eczema or psoriasis.
7. Treatment or history of any type of cancer.
8. Individuals who were under treatment for asthma or diabetes.
9. Individuals with a known sensitivity to cosmetics or personal care products.
10. Individuals who had not been on any face for the past 14 days.
11. Individuals with ANY facial piercings.
12. Individuals with any facial or neck tattoos (with the exception of thin liner on the upper lash line [no winged eyeliner]).
13. Individuals who were not willing to remove jewelry (earrings) prior to photography.

Test Procedure

The study was an 8-week study, preceded by a 1-week washout period. During the test period, the test product was used by each of the subjects according to the use instructions.

Subjects reported to the Testing Facility to begin the washout phase. Subjects were qualified by the trained technician before filling out paperwork. Once qualified, subjects filled out paperwork and were instructed to wash their face with Cetaphil® soap for 1 week.

Following the 1-week washout period, subjects reported to the Testing Facility for the baseline visit with no personal care products or cosmetics on their face. An irritation evaluation was performed on the face for safety purposes by the trained technician. The trained technician took digital images of each subject using the Visia CR® Imaging System (Canfield Scientific, Fairfield, N.J.). Using lmagePro® software (MediaCybernetics, Bethesda, Md.) the images were analyzed to determine changes (if any) in fine lines and wrinkles on the forehead, crow's feet fine lines/wrinkles, and skin plumpness.

The trained technician also took a Cutometer® (Courage+Khazaka, Germany) measurement on the face (to measure firmness and elasticity of the skin) of each subject. Subjects completed the first application of the test product at the Testing Facility, under the supervision of a trained technician. Immediately following application, all measurements were repeated including Visia pictures and professional photography. Subjects answered a questionnaire. Subjects then were given the test product and daily dairy.

Subjects returned to the Testing Facility after 1, 4 and 8 weeks of use for additional photographs, and Cutometer® measurements. At each visit, subjects answered a questionnaire.

Baseline Evaluations

All subjects reported to the Testing Facility for the baseline visit with a freshly washed “clean face” (without wearing face/eye area cosmetics or having applied any skin care products to the face). Subjects were evaluated for qualification by a trained technician.

At the baseline visit, visual evaluations and digital photographs, Cutometer® measurements were taken. Evaluations were conducted according to the procedures.

Subjects were given the test product to take home and a daily diary with the following instructions:

Instructions:

The following must be included in this diary:
1. The date and times (AM and PM) product was used.
2. Any comments or observations you may have had while using the product.
3. DO NOT USE ANY OTHER SERUM OR NEW SKIN CARE/COSMETIC PRODUCTS DURING THE TEST PROCEDURE.
4. Apply the product according to the directions below:

Directions:

After cleansing skin, apply a thin layer directly to lines & wrinkles AM and PM. Follow with the next step in your skincare regimen. Apply a sunscreen of SPF15 or higher during the day while using this product.

Test Procedure (Continued)

One-, Four- and Eight-Week Evaluations

Subjects were instructed to return to the Testing Facility after 1, 4 and 8 weeks of product use for additional digital photographs, irritation evaluations, and Cutometer® measurements. All subjects answered a questionnaire at each visit.

Clinical Evaluation Procedures

Evaluations for all parameters were conducted according to the scales and procedures outlined below.

Evaluation of Lines and Wrinkles on the Forehead

At the baseline visit only (for qualifications purposes) a trained technician evaluated fine lines and wrinkles on the forehead of each subject according to the scale below.

Scale for Scoring Fine Lines/Wrinkles on the Forehead

0=No fine lines/wrinkles

1-3=Slight fine lines/wrinkles

4-6=Noticeable fine line/wrinkles

7-9=Very noticeable fine lines/wrinkles

Evaluation of Crow's Feet Fine Lines/Wrinkles

At the baseline visit only (for qualifications purposes) a trained technician evaluated the appearance crow's feet fine lines/wrinkles on each subject according to the scale below:

Scale for Scoring Crow's Feet Fine Lines/Wrinkles

0=No fine lines

1-3=Slight fine lines

4-6=Noticeable fine lines

7-9=Very noticeable fine lines

Evaluation of Skin Plumpness

At the baseline visit only (for qualification purposes), a trained technician evaluated skin plumpness on the face of each subject according to the scale below:

Scale for Scoring Skin Plumpness

0=Very noticeable skin plumpness

1-3=Noticeable skin plumpness

4-6=Slight skin plumpness

7-9=No skin plumpness/flat appearance

Evaluation of Irritation

At baseline, immediately post-application and after 1, 4 and 8 weeks, a trained technician evaluated the face of each subject for irritation according to the scale below.

This evaluation was for safety purposes only and was not used in determining efficacy.

Scale for Scoring irritation

0=No irritation present

+=Barely perceptible irritation present

1=Mild irritation present

2=Moderate irritation present

3=Marked irritation present

4=Severe Irritation present

Digital Photography Procedure and Analysis

At baseline, immediately after application, and at 1, 4, and 8 weeks, digital images of the face were taken from the front, right and left views. At each evaluation, digital images of the face of each subject were taken from the front, right and left views using the Visia CR® 2.2 (Canfield Scientific, Fairfield, N.J.). In order to ensure consistency between the photographs, each subject wore a black t-shirt or will be draped with a disposable black cloth around the shoulders to eliminate the appearance of clothing in the pictures and each subject wore a black headband to pull hair off of and away from the face. The images were analyzed using Image Pro® software (MediaCybernetics, Bethesda, Md.) to determine changes (if any) in the appearance of: fine lines and wrinkles on the forehead, crow's feet fine lines/wrinkles, and skin plumpness.

Fine Lines and Wrinkles on the Forehead—Image Analysis

Fine lines and wrinkling in the skin are detected using either Standard lighting or a fixed angle directional light source (Cross polarized light flashes) at fixed angles and then analyzed as the Mean Area Score. A decrease in this score represents an improvement in the appearance of fine lines & wrinkles on the forehead.

Crow's Fine Lines and Wrinkles—Image Analysis

Fine lines and wrinkling in the skin are detected using either Standard lighting or a fixed angle directional light source (Cross polarized light flashes) at fixed angles and then analyzed as the Mean Area Score. A decrease in this score represents an improvement in the appearance of crow's fine lines & wrinkles.

Skin Plumpness—Image Analysis

In order to determine changes in skin plumpness, the width of all wrinkles present on the face was measured. A decrease in the mean wrinkle score represented an improvement. An increase represented a worsening

Cutometer® Measurements

At baseline, immediately after the first application, and at 1, 4, and 8 weeks, a trained technician took Cutometer® measurements. The elasticity of the skin was measured on the face of each subject using the Cutometer® R2 parameter. An increase in the Cutometer® R2 measurements indicated an improvement (increase) in skin elasticity. A decrease represented a worsening.

The firmness of the skin was measured on the face of each subject using the Cutometer® RO parameter. A decrease in the Cutometer® RO measurements indicated an improvement (increase) in skin firmness. An increase represented a worsening.

Consumer Perception Evaluation

At each visit, subjects completed a questionnaire. When responding to the questionnaires, subjects were supplied with a full-face mirror to examine their face while responding to the questions.

Statistical Analysis

All data points collected were compared to the baseline of each subject for differences between the time points. Data collected from discontinued subjects may not have been included in the statistical analysis.

The summation of the difference was analyzed using the Wilcoxon Signed-Rank Test. A response was considered a statistically significant difference from baseline when the p-value was <0.05.

Results and Discussion

Thirty-three (33) female subjects between the ages of 44 and 65 years were empanelled for the study. A total of 31 subjects (31/33) subjects successfully completed the study. Subject Nos. 27 and 33 were discontinued due to reasons unrelated to the conduct of the study.

Forehead Fine Lines/Wrinkles—Image Analysis

At baseline, immediately after the first application, and after 1, 4, and 8 weeks of product use, a trained technician took digital images of the face of each subject with the Visia CR® imaging system. Using lmagePro® software, the images were analyzed to determine changes (if any) in the appearance of forehead fine lines/wrinkles. A decrease in the mean area score represents an improvement (or decrease) in the appearance of forehead fine lines/wrinkles.

The following table presents a summary of the forehead fine lines/wrinkles image analysis.

TABLE 4
Forehead Fine Lines/Wrinkles - Image Analysis
Mean Score ± Standard Deviation (S.D), Mean Change from
Baseline And % of Subjects with Improvement from Baseline

| | | | Mean | % of subjects |
|---|---|---|---|---|
| | Mean | | Change from | with Improvement |
| | Score ± S.D. | p-value | Baseline | from Baseline |
| Baseline | 773.1 ± 190.7 | — | — | — |
| Immediate | 700.9* ± 170.6 | <0.001 | −9.3% | 97% |
| 1 Week | 663.3* ± 142.2 | <0.001 | −14.2% | 100% |
| 4 Weeks | 590.1* ± 146.6 | <0.001 | −23.7% | 100% |
| 8 Weeks | 569.6* ± 112.2 | <0.001 | −26.3% | 97% |

*Statistically significant compared with baseline, p ≤ 0.05

When images taken immediately after the first application and after 1, 4, and 8 weeks of product use were compared with baseline images, there were mean improvements of 9.3%, 14.2%, 23.7%, and 26.3%, respectively, based on image analysis. The improvements were highly significant compared with baseline. A total of 97% 100%, 100%, and 97% of the subjects showed improvement immediately after first application and after 1, 4, and 8 weeks of use, respectively.

Crow's Feet Fine Lines/Wrinkles—Image Analysis

At baseline, immediately after the first application, and after 1, 4, and 8 weeks of product use, a trained technician took digital images of the face of each subject with the Visia CR® imaging system. Using lmagePro® software, the images were analyzed to determine changes (if any) in the appearance of crow's feet fine lines/wrinkles. A decrease in the mean area score represents an improvement (or decrease) in the appearance of crow's feet fine lines/wrinkles. The following table presents a summary of the crow's feet fine lines/wrinkles image analysis.

TABLE 5
Crow's Feet Lines/Wrinkles - Image Analysis
Mean Score ± S.D., Mean Change from Baseline and %
of Subjects with Improvement from Baseline

| | | | Mean | % of Subjects |
|---|---|---|---|---|
| | Mean | | Change from | with Improvement |
| | Score ± S.D. | p-value | Baseline | from Baseline |
| Baseline | 781.0 ± 174.8 | — | — | — |
| Immediate | 691.0* ± 121.3 | <0.001 | −11.5% | 94% |
| 1 Week | 626.6* ± 108.6 | <0.001 | −19.8% | 100% |
| 4 Weeks | 553.5* ± 92.4 | <0.001 | −29.1% | 94% |
| 8 Weeks | 533.8* ± 82.9 | <0.001 | −31.7% | 100% |

*Statistically significant compared with baseline, p ≤ 0.05

When images taken immediately after first application and after 1, 4, and 8 weeks of product use were compared with baseline images, there were mean improvements of 11 0.5%, 19.8%, 29.1%, and 31 0.7%, respectively, based on image analysis. The improvements observed were highly significant compared with baseline. A total of 94%, 100%, 94%, and 100% of the subjects showed improvement immediately after first application and after 1, 4, and 8 weeks of use, respectively.

Skin Plumpness—Image Analysis

At baseline, immediately after the first application and after 1, 4, and 8 weeks of use, a trained technician took digital images of each subject with the Visia CR® imaging system. Using lmagePro® Software, skin evenness was analyzed to determine changes in skin plumpness. A decrease in the score indicates an improvement. The following table presents a summary of the skin plumpness image analysis.

TABLE 6
Skin Plumpness - Image Analysis
Mean Score ± S.D., Mean Change from Baseline and %
of Subjects with Improvement from Baseline

| | | | Mean | % of Subjects |
|---|---|---|---|---|
| | Mean | | Change from | with Improvement |
| | Score ± S.D. | p-value | Baseline | from Baseline |
| Baseline | 19.1 ± 2.0 | — | — | — |
| Immediate | 18.3* ± 1.4 | 0.009 | −4.2% | 68% |
| 1 Week | 18.1* ± 1.8 | 0.003 | −5.2% | 68% |
| 4 Weeks | 16.6* ± 1.4 | <0.001 | −13.1% | 97% |
| 8 Weeks | 16.6* ± 1.4 | <0.001 | −13.1% | 97% |

*Statistically significant difference from baseline, p ≤ 0.05

When images taken immediately after the first application and after 1, 4, and 8 weeks of product use were compared with baseline images, there were mean improvements of 4.2%, 5.2%, 13.1%, and 13.1%, respectively, based on image analysis. The improvements observed were statistically significant compared with baseline. A total of 68%, 68%, 97%, and 97% of the subjects showed improvement immediately post-application and after 1, 4, and 8 weeks of use, respectively.

Cutometer® RO Evaluation

At baseline, immediately after application, and after 1, 4, and 8 weeks of product use, a trained technician took Cutometer® RO measurements on the face of each subject to measure the firmness of the skin. A decrease in the measurements indicates an improvement (increase) in skin firmness.

TABLE 7
Cutometer ® R0 Measurements
Mean ± S.D., Mean Change from Baseline and %
of Subjects with Improvement from Baseline

| | | | Mean | |
|---|---|---|---|---|
| | | | Change | % of Subjects |
| | | | from | with Improvement |
| Mean ± S.D. | | p-value | Baseline | from Baseline |
| 0.182 ± 0.058 | Baseline | — | — | — |
| 0.194 ± 0.047 | Immediate | <0.605 | 6.6% | 52% |
| 0.220 ± 0.076 | 1 Week | <0.071 | 20.9% | 33% |
| 0.161 ± 0.044 | 4 Weeks | <0.118 | −11.5% | 61% |
| 0.096* ± 0.057 | 8 Weeks | <0.001 | −47.3% | 84% |

*Statistically significant difference from baseline, p ≤ 0.05
**For the 1-week visit, 4 subjects are not presented. This is based on 27 subjects in total.

When measurements taken immediately after application and after 1, 4, and 8 weeks of product use were compared with baseline measurements, there were mean worsenings of 6.6% and 20.9% and mean improvements of 11 0.5% and 47.3%, respectively, based on Cutometer® RO measurements. The improvement was highly significant compared with baseline at the 8-week visit. A total of 52%, 33%, 61%, and 84% of the subjects showed improvement immediately after application and after 1, 4, and 8 weeks of use, respectively.

Cutometer® R2 Evaluation

At baseline, immediately after application, and after 1, 4, and 8 weeks of product use, a trained technician took Cutometer® R2 measurements on the face of each subject to measure the elasticity of the skin. An increase in the measurements indicates an improvement in skin elasticity. The following table presents a summary of Cutometer® R2 evaluation.

TABLE 8
Cutometer ® R2 Measurements
Mean ± S.D., Mean Change from Baseline and %
of Subjects with Improvement from Baseline

| | | | Mean | % of Subjects |
|---|---|---|---|---|
| | | | Change from | with Improvement |
| | Mean ± S.D. | p-value | Baseline | from Baseline |
| Baseline | 0.408 ± 0.086 | — | — | — |
| Immediate | 0.505* ± 0.099 | <0.001 | 23.8% | 94% |
| 1 Week | 0.509* ± 0.091 | <0.001 | 24.8% | 93% |
| 4 Weeks | 0.523* ± 0.105 | <0.001 | 28.2% | 100% |
| 8 Weeks | 0.536* ± 0.101 | <0.001 | 31.4% | 100% |

*Statistically significant difference from baseline, p ≤ 0.05
**For the 1-week visit, 4 subjects are not presented. This is based on 27 subjects in total.

When measurements taken immediately after application and after 1, 4, and 8 weeks of product use were compared with baseline measurements, there were mean improvements of 23.8%, 24.8%, 28.2%, and 31.4%, respectively, based on Cutometer® R2 measurements. The changes were highly significant compared with baseline. A total of 94%, 93%, 100%, and 100% of the subjects showed improvement immediately after application and after 1, 4, and 8 weeks of use, respectively.

Skin Irritation—Technician Evaluation

At baseline, immediately after the first use and after 1, 4, and 8 weeks of product use, a trained technician evaluated the face of each subject for irritation.

TABLE 9
Skin Irritation - Technician Evaluation
Mean Score., Mean Change from Baseline and %
of Subjects with Change from Baseline

| | Mean | Mean Change | % of Subjects with |
|---|---|---|---|
| | Score | from Baseline | Change from Baseline |
| Baseline | 0.0 | — | — |
| Immediate | 0.0 | 0% | 0% |
| 1 Week | 0.0 | 0% | 0% |
| 4 Weeks | 0.0 | 0% | 0% |
| 8 Weeks | 0.0 | 0% | 0% |

There was no irritation observed on any subject during the course of the study.

Questionnaire Response

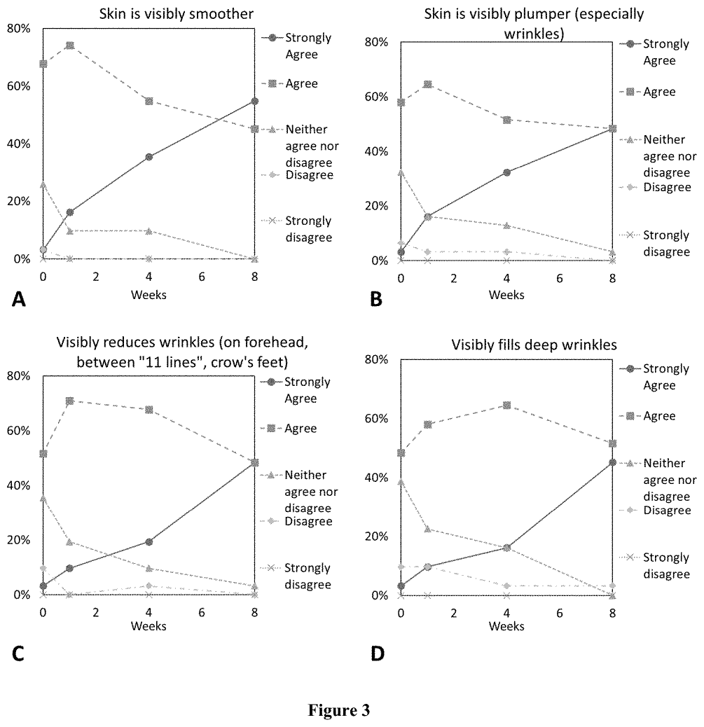

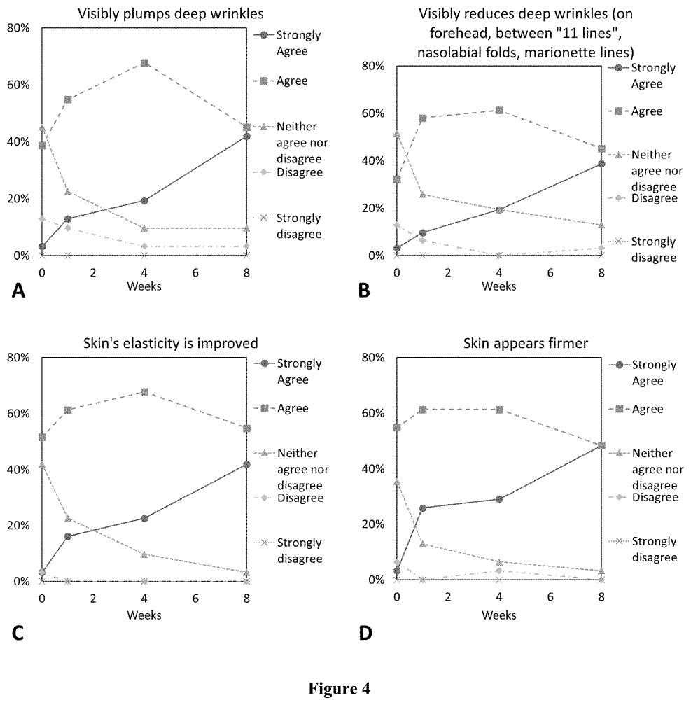

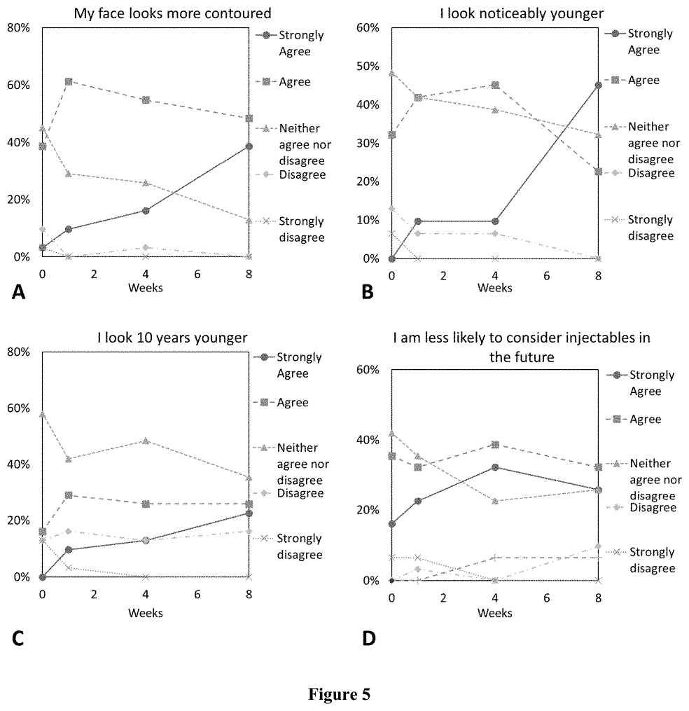

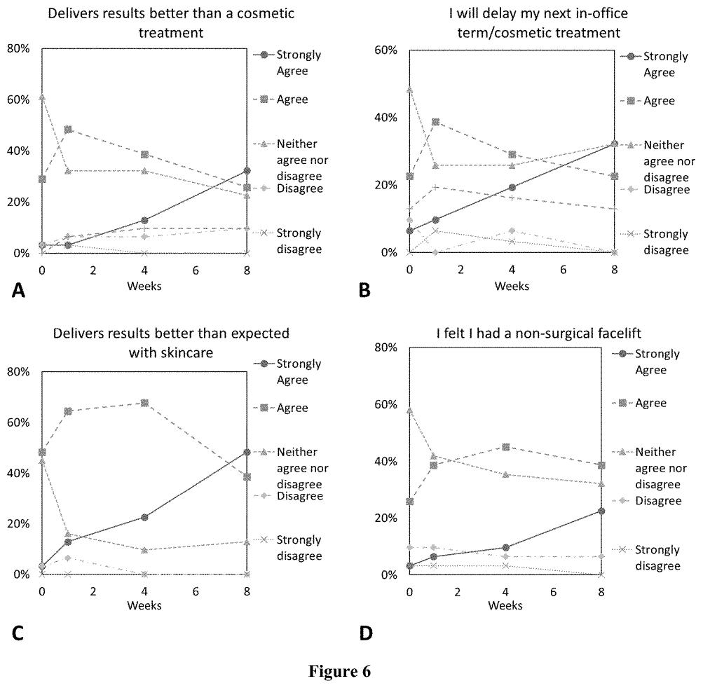

Subjects were required to complete a questionnaire immediately after application and after 1 week, 4 weeks and 8 weeks of product use. FIGS. 3-6 summaries the responses to the questionnaire with timepoint 0 corresponding to immediately after the first application.

Immediately after application, subjects were required to complete a questionnaire. The following table presents a summary of questionnaire responses for the test product.

TABLE 10
Questionnaire Responses - Immediately After Application

| | Strongly | Neither | Disagree |
|---|---|---|---|
| | Agree or | Agree nor | or Strongly |
| | Agree | Disagree | Disagree |
| Skin is visibly smoother. | 71% | 26% | 3% |
| Skin is visibly plumper (especially | 61% | 32% | 6% |
| wrinkles) | | | |
| Visibly reduces winkles (on | 55% | 35% | 10% |
| forehead, between “11 Lines”, | | | |
| crow's feet). | | | |
| Visibly fills deep wrinkles. | 52% | 39% | 10% |
| Visibly plumps deep wrinkles | 42% | 45% | 13% |
| Visibly reduces deep wrinkles (on | 35% | 52% | 13% |
| forehead, between “11 lines” | | | |
| nasolabial folds, marionette lines) | | | |
| Skin's elasticity is improved. | 55% | 42% | 3% |
| Skin appears firmer. | 58% | 35% | 6% |
| My face looks more contoured. | 42% | 45% | 13% |
| I look noticeably younger. | 32% | 48% | 19% |
| I look 10 years younger | 16% | 58% | 26% |

| | Strongly | Neither | Disagree | |
|---|---|---|---|---|
| | Agree | Agree | or | |
| | or | nor | Strongly | |
| | Agree | Disagree | Disagree | N/A |
| I am less likely to consider | 52% | 42% | 6% | 0% |
| injectable in the future. | | | | |
| Delivers results better than | 32% | 61% | 6% | 0% |
| a cosmetic treatment. | | | | |
| I will delay my next in-office | 29% | 48% | 10% | 13% |
| term/cosmetic treatment. | | | | |

| | Strongly | Neither | Disagree |
|---|---|---|---|
| | Agree or | Agree nor | or Strongly |
| | Agree | Disagree | Disagree |
| Delivers results better than | 52% | 45% | 3% |
| expected with skincare. | | | |
| I felt I had a non-surgical facelift. | 29% | 58% | 13% |
| The package was easy to use and | 87% | 13% | 0% |
| maneuver. | | | |
| The package targeted my lines and | 61% | 29% | 10% |
| wrinkles effectively. | | | |
| The package was more innovative | 65% | 32% | 3% |
| than any other skincare product I | | | |
| have tried before. | | | |
| It dispensed the right amount. | 97% | 3% | 0% |

A majority of subjects (52%-97%) responded positively to some of the questions about various attributes of the product immediately after the first use. A total of 97% of the subjects strongly agreed or agreed that the product dispensed the right amount.

After 1 week of product use, subjects were required to complete a questionnaire. The following table presents a summary of questionnaire responses for the test product.

TABLE 11
Questionnaire Responses - 1 Week

| | Strongly | Neither | Disagree |
|---|---|---|---|
| | Agree or | Agree nor | or Strongly |
| | Agree | Disagree | Disagree |
| Skin is visibly smoother. | 90% | 10% | 0% |
| Skin is visibly plumper (especially | 81% | 16% | 3% |
| wrinkles) | | | |
| Visibly reduces winkles (on | 81% | 19% | 0% |
| forehead, Between “11 Lines”, | | | |
| crow's feet). | | | |
| Visibly fills deep wrinkle. | 68% | 23% | 10% |
| Visibly plumps deep wrinkles | 68% | 23% | 10% |
| Visibly reduces deep wrinkles (on | 68% | 26% | 6% |
| forehead, between “11 lines” | | | |
| nasolabial folds, marionette lines) | | | |
| Skin's elasticity is improved. | 77% | 23% | 0% |
| Skin appears firmer. | 87% | 13% | 0% |
| My face looks more contoured. | 71% | 29% | 0% |
| I look noticeably younger. | 52% | 42% | 6% |
| I look 10 years younger | 38% | 42% | 19% |

| | Strongly | Neither | Disagree | |
|---|---|---|---|---|
| | Agree | Agree | or | |
| | or | nor | Strongly | |
| | Agree | Disagree | Disagree | N/A |
| I am less likely to consider | 55% | 35% | 10% | 0% |
| injectable in the future. | | | | |
| Delivers results better than | 52% | 32% | 10% | 6% |
| a cosmetic treatment. | | | | |
| I will delay my next in-office | 48% | 26% | 6% | 19% |
| term/cosmetic treatment. | | | | |

| | Strongly | Neither | Disagree |
|---|---|---|---|
| | Agree or | Agree nor | or Strongly |
| | Agree | Disagree | Disagree |
| Delivers results better than | 77% | 16% | 6% |
| expected with skincare. | | | |
| I felt I had a non-surgical facelift. | 45% | 42% | 13% |
| The package was easy to use and | 90% | 6% | 3% |
| maneuver. | | | |
| The package targeted my lines and | 84% | 16% | 0% |
| wrinkles effectively. | | | |
| The package was more innovative | 90% | 6% | 3% |
| than any other skincare product I | | | |
| have tried before. | | | |
| It dispensed the right amount. | 97% | 3% | 0% |

A majority of subjects (52%-97%) responded positively to most questions about various attributes of the product after 1 week of product use. A total of 97% of the subjects strongly agreed or agreed that the product dispensed the right amount.

After 4 weeks of product use, subjects were required to complete a questionnaire. The following table presents a summary of questionnaire responses for the test product.

TABLE 12
Questionnaire Responses - 4 Weeks

| | Strongly | Neither | Disagree |
|---|---|---|---|
| | Agree or | Agree nor | or Strongly |
| | Agree | Disagree | Disagree |
| Skin is visibly smoother. | 90% | 10% | 0% |
| Skin is visibly plumper (especially | 84% | 13% | 3% |
| wrinkles) | | | |
| Visibly reduces winkles (on | 87% | 10% | 3% |
| forehead, between“11 Lines” | | | |
| crow's feet). | | | |
| Visibly fills deep wrinkles | 81% | 16% | 3% |
| Visibly plumps deep wrinkles | 87% | 10% | 3% |
| Visibly Reduces deep wrinkles (on | 81% | 19% | 0% |
| forehead, between “11 lines” | | | |
| nasolabial folds, marionette lines) | | | |
| Skin's elasticity is improved. | 90% | 10% | 0% |
| Skin appears firmer. | 90% | 6% | 3% |
| My face looks more contoured. | 71% | 26% | 3% |
| I look noticeably younger. | 55% | 39% | 6% |
| I look 10 years younger | 39% | 48% | 13% |

| | Strongly | Neither | Disagree | |
|---|---|---|---|---|
| | Agree or | Agree nor | or Strongly | |
| | Agree | Disagree | Disagree | N/A |
| I am less likely to consider | 71% | 23% | 0% | 6% |
| injectable in the future. | | | | |
| Delivers results better than | 52% | 32% | 6% | 10% |
| a cosmetic treatment. | | | | |
| I will delay my next term/ | 48% | 26% | 10% | 16% |
| cosmetic treatment. | | | | |

| | Strongly | Neither | Disagree |
|---|---|---|---|
| | Agree or | Agree nor | or Strongly |
| | Agree | Disagree | Disagree |
| Delivers results better than | 90% | 10% | 0% |
| expected with skincare. | | | |
| I felt I had a non-surgical facelift | 55% | 35% | 10% |
| The package was easy to use and | 100% | 0% | 0% |
| maneuver. | | | |
| The package targeted my lines and | 94% | 6% | 0% |
| wrinkles effectively | | | |
| The package was more innovative | 90% | 6% | 3% |
| than any other skincare product I | | | |
| have tried before. | | | |
| It dispensed the right amount. | 94% | 3% | 3% |

A majority of subjects (52%-100%) responded positively to most questions about various attributes of the product after 4 weeks of product use. All of the subjects strongly agreed or agreed the package was easy to use and maneuver.

After 8 weeks of product use, subjects were required to complete a questionnaire. The following table presents a summary of questionnaire responses for the test product.

TABLE 13
Questionnaire Responses - 8 Weeks

| | Strongly | Neither | Disagree |
|---|---|---|---|
| | Agree or | Agree nor | or Strongly |
| | Agree | Disagree | Disagree |
| Skin is visibly smoother. | 100% | 0% | 0% |
| Skin is visibly plumper (especially | 97% | 3% | 0% |
| wrinkles) | | | |
| Visibly reduces winkles (on | 97% | 3% | 0% |
| forehead, between “11 Lines” | | | |
| crow's feet). | | | |
| Visibly fills deep wrinkles. | 97% | 0% | 3% |
| Visibly plumps deep wrinkles. | 87% | 10% | 3% |
| Visibly Reduces deep wrinkles (on | 85% | 12% | 3% |
| forehead, between “11 lines” | | | |
| nasolabial folds, marionette lines). | | | |
| Skin's elasticity is improved. | 97% | 3% | 0% |
| Skin appears firmer. | 97% | 3% | 0% |
| My face looks more contoured. | 87% | 13% | 0% |
| I look noticeably younger. | 68% | 32% | 0% |
| I look 10 years younger. | 48% | 35% | 16% |

| | Strongly | Neither | Disagree | |
|---|---|---|---|---|
| | Agree | Agree | or | |
| | or | nor | Strongly | |
| | Agree | Disagree | Disagree | N/A |
| I am less Likely to consider | 58% | 26% | 10% | 6% |
| injectable in the future. | | | | |
| Delivers results better than | 58% | 23% | 10% | 10% |
| a cosmetic treatment. | | | | |
| I will delay my next term/ | 55% | 32% | 0% | 13% |
| cosmetic treatment. | | | | |

| | Strongly | Neither | Disagree |
|---|---|---|---|
| | Agree or | Agree nor | or Strongly |
| | Agree | Disagree | Disagree |
| Delivers results better than | 87% | 13% | 0% |
| expected with skincare. | | | |
| I felt I had a non-surgical facelift. | 61% | 32% | 6% |
| The package was easy to use and | 97% | 3% | 0% |
| maneuver. | | | |
| The package targeted my lines ad | 97% | 3% | 0% |
| wrinkles effectively | | | |
| The package was more innovative | 94% | 6% | 0% |
| than any other skincare product I | | | |
| have tried before. | | | |
| It dispensed the right amount. | 100% | 0% | 0% |

A majority of subjects (58%-100%) responded positively to almost all questions about various attributes of the product after 8 weeks of product use. All of the subjects strongly agreed or agreed the package dispensed the right amount.

Summary and Conclusions

A clinical efficacy and consumer perception study was conducted with 31 subjects to determine if the cosmetic composition according to Example 3 improved various parameters of the skin.

Skin Plumpness was significantly improved immediately post-application and after 1, 4, and 8 weeks of product use, based on image analysis.

Crow's Feet Fine Lines/Wrinkles were significantly improved immediately post-application and after 1, 4, and 8 weeks of product use, based on image analysis.

Forehead Fine Lines/Wrinkles were significantly improved immediately post-application and after 1, 4, and 8 weeks of product use, based on image analysis.

Skin Firmness was significantly improved after 8 weeks of product use, based on Cutometer measurements.

Skin Elasticity was significantly improved immediately post-application and after 1, 4, and 8 weeks of product use, based on Cutometer measurements.

The product did not cause any irritation on any subject during the course of the study.

The product was associated with a moderately low level of acceptance immediately after their first use, a moderate level of acceptance after 1 week of product use and moderately high level of subject acceptance after 4 and 8 weeks of use, with a majority of subjects responding positively to all questions about various attributes of product performance.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A cosmetic composition comprising Swertia chirata extract, hyaluronic acid, and retinoid.

2. The cosmetic composition of claim 1, wherein the composition comprises about 0.005 wt. % to about 0.500 wt. % Swertia chirata extract.

3. The cosmetic composition of claim 1, wherein the composition comprises about 0.005 wt. % to about 1.00 wt. % retinoid.

4. The cosmetic composition of claim 1, wherein the composition comprises about 0.001 wt. % to about 2.0 wt. % hyaluronic acid.

5. The cosmetic composition of claim 1, wherein the composition comprises about 0.005 wt. % to about 0.500 wt. % Swertia chirata extract; about 0.005 wt. % to about 1.00 wt. % retinoid; and about 0.001 wt. % to about 2.0 wt. % hyaluronic acid.

6. The cosmetic composition of claim 1, wherein the composition comprises about 0.030 wt. % Swertia chirata extract; about 0.070 wt. % retinoid; and about 0.050 wt. % hyaluronic acid.

7. The cosmetic composition of claim 1, wherein the composition comprises about 0.030 wt. % Swertia chirata extract; about 0.110 wt. % retinoid; and about 0.002 wt. % hyaluronic acid.

8. The cosmetic composition of claim 1, wherein the retinoid is selected from the group consisting of retinol, retinal, tretinoin, isotretinoin, alitretinoin, etretinate acitretin, adapalene, bexarotene, and tazarotene, and trifarotene.

9. The cosmetic composition of claim 8, wherein the retinoid is retinol.

10. The cosmetic composition claim 1, wherein the hyaluronic acid comprises a mixture of hyaluronic acids of different molecular weights.

11. The cosmetic composition of claim 10, wherein the mixture comprises at least three hyaluronic acids of different molecular weights.

12. The cosmetic composition of claim 1, further comprising a vitamin C compound.

13. The cosmetic composition of claim 12, wherein the vitamin C compound comprises ascorbic acid or 3-O-ethyl ascorbic acid.

14. The cosmetic composition of claim 1, further comprising a chromenone of formula (I): 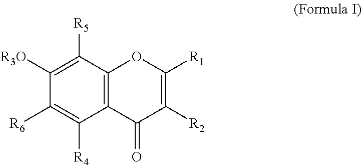
or a salt thereof; wherein:
R¹ and R² are identical or different, and are selected from the group consisting of H, —(C═O)—R⁷, —C(═O)—OR⁷, a straight-chain or branched C₁- to C₂₀-alkyl group, wherein the alkyl is optionally at least once interrupted by oxygen, a straight-chain or branched C₃- to C₂₀-alkenyl group, a straight-chain or branched to C₂₀-hydroxyalkyl or di- or polyhydroxyalkyl group, where the hydroxyl group is bonded to a primary or secondary carbon atom of the alkyl, and wherein the alkyl is optionally at least once interrupted by oxygen, a C₃- to C₁₀-cycloalkyl group and a C₃- to C₁₂-cycloalkenyl group (where the cyclic group is optionally bridged by —CH₂)ₙ— group where n=1 to 3);
R³ is H or a straight-chain or branched C₁- to C₂₀-alkyl group;
R⁴ is H or —OR⁸;
R⁵ and R⁶ are identical or different, and are selected from the group consisting of H or —OH, a straight-chain or branched C₁- to C₂₀-alkyl group (wherein the alkyl is optionally at least once interrupted by oxygen), a straight-chain or branched C₃- to C₂₀-alkenyl group, and a straight-chain or branched C₁- to C₂₀-hydroxyalkyl group, where the hydroxyl group is bonded to a primary or secondary carbon atom of the alkyl, and wherein the alkyl is optionally at least once interrupted by oxygen;
R⁷ is selected from the group consisting of H, a straight-chain or branched C₁- to C₂₀-alkyl group, wherein the alkyl is optionally at least once interrupted by oxygen, a straight-chain or branched C₃- to C₂₀-alkenyl group, and a straight-chain or branched C₁- to C₂₀-hydroxy-alkyl or -di- or polyhydroxyalkyl group, where the hydroxyl group is bonded to a primary or secondary carbon atom of the alkyl and wherein the alkyl is optionally at least once interrupted by oxygen; and
R⁸ is H or a straight-chain or branched C₁- to C₂₀-alkyl group.

15. The cosmetic composition of claim 14, wherein the cosmetic composition comprises about 0.1 wt. % to about 2.0 wt. % of a compound of formula (I).

16. The cosmetic composition of claim 14, wherein the compound of formula (I) is dihydroxy methylchromenone.

17. The cosmetic composition of claim 1, wherein the cosmetic composition comprises at least 60 wt. % water.

18. The cosmetic composition of claim 1, wherein the cosmetic composition is a cream or a serum.

19. A method of reducing fine lines and/or wrinkles, and/or smoothing the skin, comprising topically administering to the skin the cosmetic composition of claim 1.

20. The method of claim 19, wherein the skin is the skin of the face and/or neck.

21. The method of claim 19, wherein the cosmetic composition is applied to the forehead, the lip, between the brows, under the eyes, the nasolabial folds, crow's feet, or a combination thereof.

22. The method of claim 19, wherein the skin is cleansed skin.

23. The method of claim 19, wherein the cosmetic composition is applied in the morning, the evening, or both.
